(19) United States
(12) Patent Application Publication
(10) Pub. No.: US 2021/0395283 A1
(43) Pub. Date: Dec. 23, 2021

(54) COMPOUND, COMPLEX, PREPARATION METHOD THEREOF, AND USE THEREOF

(71) Applicant: JIANGSU OXIRANCHEM CO., LTD, Yizheng (CN)

(72) Inventors: Jianmin Zhu, Yizheng (CN); Zhaobin Liu, Yizheng (CN); Zhenpeng Dong, Yizheng (CN)

(21) Appl. No.: 17/053,820

(22) PCT Filed: Apr. 29, 2019
(86) PCT No.: PCT/CN2019/084903

§ 371 Date: Apr. 29, 2021

(30) Foreign Application Priority Data
May 15, 2018 (CN) 201810462139.6

(51) Int. Cl. C07F 15/00; C07F 15/02; C07F 15/04; C07D 307/92; C08F 10/02; C08F 4/70

(52) U.S. Cl. CPC C07F 15/0066; C07F 15/025; C07F 15/045; C08F 4/7003; C08F 10/02; C08F 4/7098; C07D 307/92

(57) ABSTRACT

The present disclosure provides a compound, a complex, a preparation method thereof, and a use thereof. The compound is represented by the following structural formula, in which R¹ to R¹⁰ are the same or different and are each independently selected from hydrogen, a hydrocarbon group having a carbon number of C₁ to C₁₆, a substituted hydrocarbon group, an alkoxy group, an alkylthio group, an alkylamino group, a haloalkylthio group, a halogen-substituted alkoxy group, a halogen-substituted alkylamino group, an aryloxy group, an arylthio group, arylamino group, a diphenylphosphino group, a halogen group, a nitro group, or a nitrile group. The complex of one embodiment of the present disclosure has a high catalytic effect, and can be used to prepare a highly branched, controllable, low molecular weight polymer with a high activity.

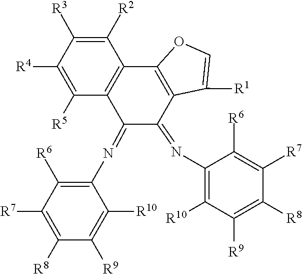

### TECHNICAL FIELD

The present disclosure relates to a compound, and particularly, to a diimine compound which can be used as ligand of a complex catalyst.

### BACKGROUND

The polymer of the most output in the world is polyolefin, which is widely used in the fields of industry, agriculture, national defense, transportation and daily necessities. Since industrialized in 1939, the polyolefin production has been constantly developed with the improvement of the catalyst technology and the polyolefin products become more high-end and varied. Therefore, the development of catalysts for olefin polymerization with excellent properties has become hot point in the research of polyolefin.

After the traditional Ziegle-Natta catalyst dominating the market for more than 30 years, the single active site catalyst production became industrialized in the mid-1990s which meant the industry of polymerization catalyst had greatly changed. Compared with the traditional Ziegle-Natta catalyst, the single active site catalyst (metallocene catalyst and non-metallocene catalyst) has technical advantages of wide applicability and easy to control, and the polymer product could be well controlled in the process of polymerization. The single active site catalyst has a single active site, therefore the polymerization product has a narrow molecular weight distribution and the monomer units in the copolymer product are distributed equally. In addition, the polymer product has good resin characteristics and high stereo-tacticity, and may be regulated in structure by molecular designing and molecular tailoring.

In 1998, Brookhart et al. reported that the alpha-diimine nickel or palladiu catalysts with large steric hindrance were highly active in catalyzing ethylene polymerization to obtain branched polyolefin (J. Am. Chem. Soc. 1995, 117, 6414-6415). The alpha-diimine catalysts have a specific structure of:

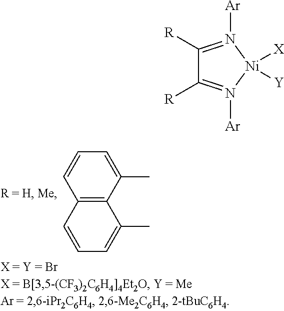

A lot of domestic researches have been made to the alpha-diimine catalysts, in which the groups in benzene ring linked to the diimine moiety are modified while the structure of 2,3-butylene diimine or acenaphthene diimine remains unchanged. In the patent applications (with application numbers of 201010572748.0, 201010572741.9, 201010593358.1 and 201310010498.5) of Northwest Normal University, acenaphthylene-1,2-dione is used and phenyl groups are introduced at two ortho-positions of the two substituted aniline structures, or groups of phenyl, phenethyl or bromine are introduced at both ortho- and para-positions of the two substituted aniline structures.

Numerous attempts have been made to the catalysts of asymmetric butylene diimine or acenaphthene diimine in the patent applications of Institute of Chemistry Chinese Academy of Sciences (with application numbers of 201410685709.X, 201310265420.8, 201110059539.0, 201410655251.3 and 201410699616.2), in which groups of benzhydryl, bis(4-fluorophenyl)methyl or phenyl are introduced at two ortho-positions of the two substituted anilines.

Similarly, two different substituted anilines are used to prepare asymmetric diimine nickel catalyst precursors in the patent application of Shanghai institute of Organic Chemistry, Chinese Academy of Sciences with an application number of 201410649300.2. One of the substituted anilines was substituted by isopropyls at two ortho-positions, and the substituted groups in the other substituted aniline were halogen, methyl and the like having small steric hindrance. The polymerization of ethylene or polar alkenes could be catalyzed with high activity to obtain high branched polymer by using the asymmetric diimine catalysts.

It can be known from analysis of the patent applications above mentioned that the activity of the catalyst and the structure of the product can be regulated mainly by adjusting electronic effect and steric effect. So far, with a larger steric hindrance caused by the substituent, especially the group at the ortho-positions of the substituted aniline, of the catalysts, the thermal stability is enhanced and the molecular weight of the polymer obtained is greater. For the nickel catalyst, the space around the central metal is relatively open, facilitating the occurrence of chain transfer reactions in the catalytic process and obtaining low branching degree and molecular weight polymer. The modification of the upper structure of the diimine structure (the 2,3-butylene diimine or acenaphthene diimine moiety) are disclosed only by a small amount of patent applications. It is disclosed in the patent applications with application numbers of 200910038504.1 and 201010177711.8 that the thermal stability of the catalyst is enhanced after changing the upper structure of the catalyst ligand diimine (2,3-dimethylbutane group or acenaphthene group) into camphorquinon group or two separate substituted phenyls. It is further disclosed in the patent application with an application number of 201510462908.9 that the catalyst has greatly enhanced thermal stability and still keeps strong catalytic activity at 100° C. after replacing the acenaphthene group of the catalyst ligand acenaphthenequinone with a complicated group of 2,3,9,10-tetraethyl-6,13-dihydro-6,13-acetylpentacene.

It is disclosed in the patent application of Zhejiang University with an application number of 201210276331.9 that the traditional acenaphthene diimine catalyst is modified by replacing acenaphthylene-1,2-dione with ethylene acenaphthylene-1,2-dione to react with a substituted aniline. The catalyst obtained has an enhanced high temperature stability and can be used to prepare hyperbranched and high molecular weight polyethylene with high activity even with an aluminum-nickel ratio of 100. The upper structure of one imine is combined with the substituted aniline moiety to form a 5-membered heterocyclic ring comprising one heteroatom selected from N, O and S besides the nitrogen atom of the imine in the patent application with an application number of 201410555075.X. The upper structure of the other imine is phenyl or substituted phenyl and the imine structure is formed by ketoamine condensation reaction of the substituted aniline. The asymmetric structure makes the catalyst used for the synthesis of highly branched and high molecular weight polyethylene.

The products of polymerization using the diimine catalysts disclosed by the patent applications above mentioned are mainly highly branched and high molecular weight polymers. It is particularly important to design the catalyst structure for preparing controllable, low molecular weight and highly branched polyethylene throughout the reaction mechanism of chain walking and at the same time taking into account the thermal stability of the catalyst.

### SUMMARY OF THE INVENTION

One object of the present disclosure is to provide a compound represented by the following structural formula:

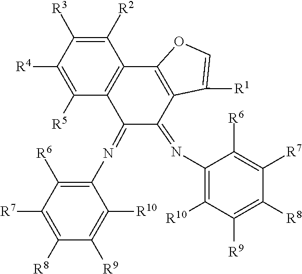

R¹ to R¹⁰ are the same or different and each independently represent a group comprising 1 to 16 carbon atoms selected from hydrocarbyl, substituted hydrocarbyl, alkoxy, alkylthio, alkylamino, haloalkylthio, haloalkoxy, haloalkylamino, aryloxy, arylthio, arylamino; or a group selected from hydrogen, diphenylphosphino, halogen, nitro, and nitrile.

One embodiment of the present disclosure provides a preparation method of the compound above mentioned comprising:

carrying out a furan cyclization reaction between a 2-hydroxy-1,4-quinone compound and a haloketone or haloaldehyde to obtain a diketone compound; and

carrying out a ketoamine condensation reaction between the diketone compound and aniline or a substituted aniline to obtain the compound.

One embodiment of the present disclosure provides a complex represented by the following structural formula:

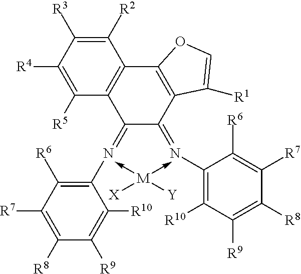

wherein M is selected from Fe, Ni or Pd;

X and Y are independently selected from halogen, C₁-C₄ alkyl or C₂-C₆ alkenyl;

R¹ to R¹⁰ are the same or different and each independently represent a group comprising 1 to 16 carbon atoms selected from hydrocarbyl, substituted hydrocarbyl, alkoxy, alkylthio, alkylamino, haloalkylthio, haloalkoxy, haloalkylamino, aryloxy, arylthio, arylamino; or a group selected from hydrogen, diphenylphosphino, halogen, nitro, and nitrile.

One embodiment of the present disclosure provides a preparation method of the complex above mentioned comprising subjecting the compound above mentioned to react with a metal salt to obtain the complex.

One embodiment of the present disclosure provides a catalyst composition comprising a main catalyst and a cocatalyst, wherein the main catalyst comprises the complex above mentioned and the cocatalyst is one or more selected from the group consisting of alkyl aluminoxane, aluminum alkyl and halogenated aluminum alkyl.

One embodiment of the present disclosure provides a use of the catalyst composition above mentioned as catalyst of olefin polymerization.

The complex of one embodiment of the present disclosure has a high catalytic effect, and can be used to prepare a highly branched, controllable and low molecular weight polymer with a high activity.

### DETAILED DESCRIPTION

Hereinafter, the representative embodiments with the features and the advantages of the present disclosure will be described in more detail. It should be understood that various changes can be made without departing from the spirit or scope of the disclosure. The descriptions and the drawings herein are only illustrative, and should not be construed as limiting in any way.

One embodiment of the present disclosure provides a compound represented by the following structural formula:

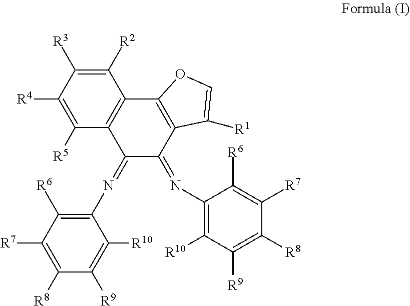

R¹ to R¹⁰ are the same or different and are each independently selected from hydrogen, hydrocarbyl, substituted hydrocarbyl, alkoxy, alkylthio, alkylamino, haloalkylthio, haloalkoxy, haloalkylamino, aryloxy, arylthio, arylamino, diphenylphosphino, halogen, nitro, or nitrile.

In one embodiment of the present disclosure, the hydrocarbyl may be saturated or unsaturated; the saturated hydrocarbyl comprises alkyl, cycloalkyl and substituted cycloalkyl; and the unsaturated hydrocarbyl may comprise aryl, substituted aryl, aralkyl and substituted aralkyl.

In one embodiment of the present disclosure, the hydrocarbyl comprises alkyl, cycloalkyl, aryl and aralkyl; and the substituted hydrocarbyl comprises haloalkyl, sulfur-containing alkyl, substituted cycloalkyl and substituted aralkyl.

In one embodiment of the present disclosure, the hydrocarbyl may be aliphatic hydrocarbon group containing 1 to 8 carbon atoms, or aryl or aralkyl containing 6 to 16 carbon atoms; and the substituted hydrocarbyl may be substituted aryl or substituted aralkyl containing 6 to 16 carbon atoms.

In one embodiment of the present disclosure, the hydrocarbyl or substituted hydrocarbyl may be alkyl, cycloalkyl, substituted cycloalkyl, aryl, substituted aryl, aralkyl and substituted aralkyl, wherein the substituents of the substituted aryl, substituted aralkyl and substituted cycloalkyl may be C₁-C₄ alkyl, C₁-C₄ haloalkyl, halogen, nitro, or nitrile.

For example, the alkyl may include methyl, ethyl and isopropyl (-iPr); the cycloalkyl may be cyclohexyl; the aryl may be phenyl; the aralkyl may include benzyl (—CH₂Ph), benzhydryl (—CH(Ph)₂); the halogen may include fluorine, chlorine, bromine and iodine; the substituted aryl may be methylphenyl (-PhCH₃); and the substituted aralkyl may be —CH₂PhBr.

In one embodiment of the present disclosure, the alkoxyl, alkylthio, sulfur-containing alkyl, haloalkylthio and alkylamino may have 1 to 4 carbon atoms, for example, methoxyl (—OCH₃), methylthio (—SCH₃) and ethylamino (—NHCH₂CH₃), and the sulfur-containing alkyl may be —CH₂SCH₃.

In one embodiment of the present disclosure, the aryloxy, arylthio and arylamino may have 6 to 16 carbon atoms, such as —OCH₂Ph, —SCH₂Ph and —NHCH₂Ph.

In one embodiment of the present disclosure, le to R⁵ are each independently selected from the following group of hydrogen, C₁-C₄ alkyl, —OR^{1′}, —SR^{2′}, —NHR^{3′}, —N(R^{4′})₂, substituted or unsubstituted phenyl, benzyl, substituted or unsubstituted cycloalkyl and halogen; and R^{1′}, R^{2′}, R^{3′}, R^{4′} are each independently selected from C₁-C₄ alkyl or C₁-C₄ haloalkyl.

Preferably, R¹ is selected from hydrogen, methyl, ethyl, isopropyl, halogen, phenyl, benzyl, benzhydryl and cycloalkyl; R² is selected from hydrogen, methyl and halogen; R³ is selected from hydrogen, methyl and halogen; R⁴ is selected from hydrogen, methyl, isopropyl, phenyl, benzyl and halogen; and R⁵ is selected from hydrogen, methyl, ethyl, isopropyl and halogen.

More preferably, R¹ is selected from hydrogen, methyl, ethyl, isopropyl, phenyl or cyclohexyl; R² is selected from hydrogen or halogen; R³ is selected from methyl, halogen or hydrogen; R⁴ is selected from methyl, isopropyl or hydrogen; and R⁵ is selected from isopropyl, methyl or hydrogen.

In one embodiment of the present disclosure, R⁶ to R¹⁰ are selected from C₁-C₈ alkyl or C₁-C₈ haloalkyl, halogen, nitro, nitrile, —OR^{1′}, —SR^{2′} or —CH₂SR^{2′}, —NHR^{3′}, —N(R^{4′})₂, phenyl, benzyl, benzhydryl and diphenylphosphino; and R^{1′}, R^{2′}, R^{3′} and R^{4′} are each independently selected from C₁-C₈ alkyl or C₁-C₈ haloalkyl and substituted or unsubstituted phenyl.

Preferably, R⁶ and R¹⁰ are selected from hydrogen, methyl, ethyl, isopropyl, phenyl, benzyl, benzhydryl and cyclohexyl; R⁷ and R⁹ are selected from hydrogen, methyl, ethyl and isopropyl; and R⁸ is selected from hydrogen, methyl, ethyl, isopropyl, phenyl, benzyl, benzhydryl and halogen.

One embodiment of the present disclosure provides a preparation method of the Formula (I) compound comprising:

carrying out a furan cyclization reaction between 2-hydroxy-1,4-quinone compound A and a haloketone or haloaldehyde to obtain a diketone compound B; and

carrying out a ketoamine condensation reaction between the compound B and compound C (aniline or a substituted aniline) to obtain the diimine compound (I).

In one embodiment of the present disclosure, the haloketone or haloaldehyde is alpha-haloketone or alpha-haloaldehyde.

In one embodiment of the present disclosure, the reaction equations of the preparation method of Formula (I) compound are as follows:

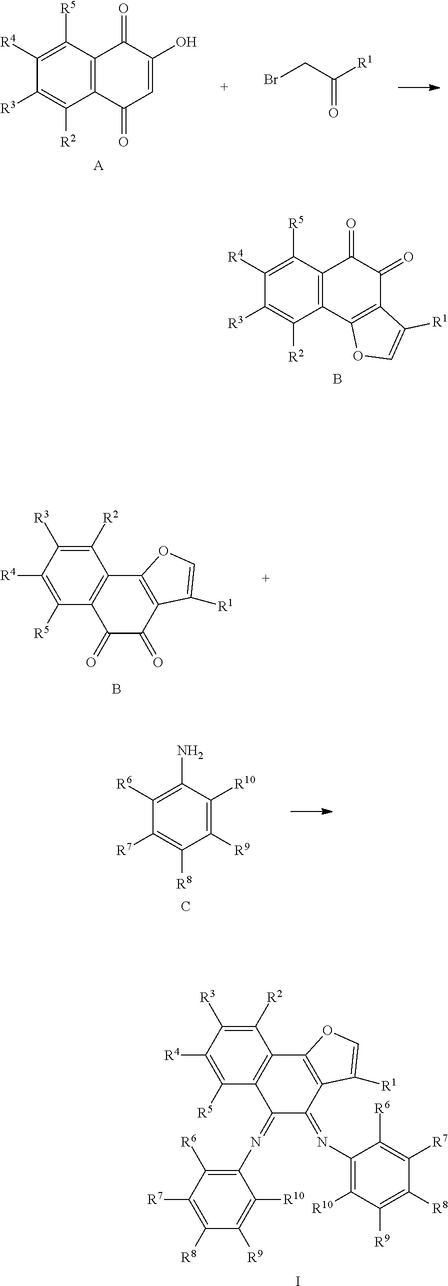

In one embodiment of the present disclosure, all of R², R³, and R⁴ are hydrogen; and the diketone compound B has a structural formula as below:

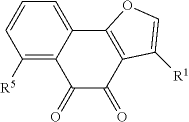

wherein R¹ may be selected from H, Me, Et, iPr, cyclohexyl, Ph, —CH₂Ph, and —CH(Ph)₂; and R⁵ may be selected from H, Me, Et and iPr.

In one embodiment of the present disclosure, in the substituted aniline C, R⁷ and R⁹ are hydrogen; R⁶ and R¹⁰ may be each independently selected from H, Me, Et, iPr, Ph, —CH₂Ph and —CH(Ph)₂; and R⁸ may be selected from H, Me, iPr, Ph, —CH₂Ph, —CH(Ph)₂ and halogen.

In one embodiment of the present disclosure, the preparation method of the Formula (I) compound comprising:

(a) subjecting a 2-hydroxy-1,4-quinone compound to a furan cyclization reaction to obtain a naphthofuran diketone compound;

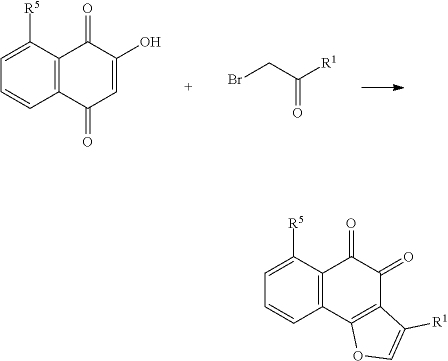

(b) carrying out a ketoamine condensation reaction between the naphthofuran diketone compound and aniline or a substituted aniline to obtain the diimine ligand L.

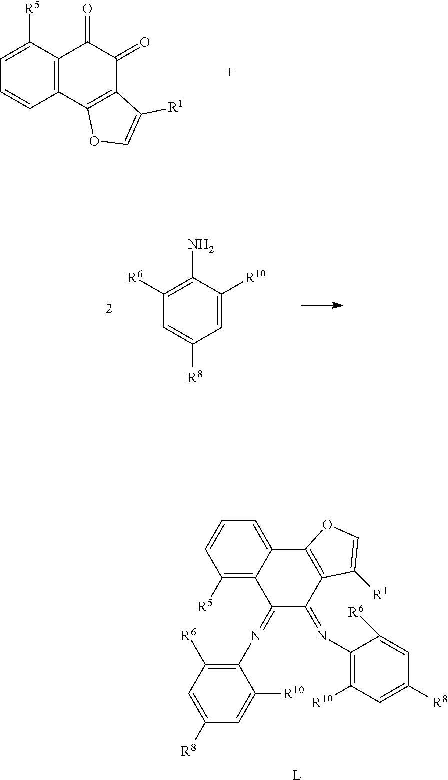

In one embodiment of the present disclosure, the furan cyclization reaction is carried out in one or more solvents selected from ethanol, petroleum ether, n-hexane, toluene and cyclohexane; the solvent is preferably ethanol, n-hexane or toluene, and more preferably ethanol or toluene.

In one embodiment of the present disclosure, the catalyst used in the furan cyclization reaction is pyridine, triethylamine or organic ammonium salt, preferably pyridine or organic ammonium salt. An example of the organic ammonium salt may be ammonium acetate.

In one embodiment of the present disclosure, the reaction temperature of the furan cyclization reaction is 10° C. to 200° C., preferably 50° C. to 180° C., more preferably 70° C. to 160° C., for example, 80° C., 100° C., 120° C. and 140° C.

In one embodiment of the present disclosure, the molar ratio of the 2-hydroxy-1,4-quinone compound to the alpha-haloketone or alpha-haloaldehyde is 1:1 to 1:10, preferably 1:2 to 1:8, for example, 1:3, 1:4, 1:5, 1:6, 1:7 and 1:7.5.

In one embodiment of the present disclosure, the molar ratio of the compound B (such as naphthofuran diketone) to the compound C (aniline or the substituted aniline) is 1:2 to 1:10, preferably 1:2 to 1:3, for example, 1:2.25, 1:2.5, 1:3.75, 1:4, 1:4.5, 1:5, 1:7 and 1:9.

In one embodiment of the present disclosure, the temperature for solvent reflux in the ketoamine condensation reaction is 40° C. to 150° C., preferably 60° C. to 120° C., for example, 70° C., 80° C., 90° C., 100° C. and 110° C.

In one embodiment of the present disclosure, the catalyst used in the ketoamine condensation reaction may be one or more selected from p-toluenesulfonic acid, acetic acid, formic acid and trifluoromethanesulfonic acid; and the catalyst has an additive amount of 0.01% to 30%, based on the mole number of the diketone compound.

In one embodiment of the present disclosure, the reaction time of the ketoamine condensation reaction is 6 h to 120 h, preferably 6 h to 48 h, for example, 10 h, 12 h, 15 h, 20 h, 30 h and 40 h.

One embodiment of the present disclosure provides a complex, prepared by the Formula (I) compound and a metal salt, having a structural formula below:

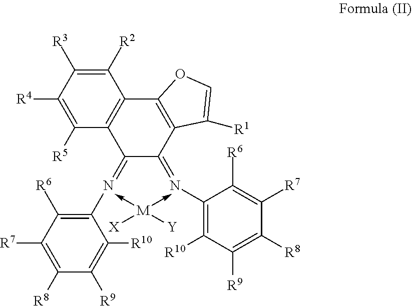

M may be Fe, Ni or Pd, preferably Ni or Pd.

X and Y are independently selected from halogen, C₁-C₄ alkyl or C₂-C₆ alkenyl, for example, methyl, ethyl, propyl, isopropyl and alkenyl (such as 1,5-cyclooctadiene); X and Y may be the same, for example, both of them are Br or C₁; or X and Y may be are different, for example, X is methyl and Y is C₁.

In one embodiment of the present disclosure, the Formula (I) compound reacts with a bivalent or trivalent metal salt in the presence of water-free and oxygen-free condition to obtain the Formula (II) complex.

In one embodiment of the present disclosure, the metal salt may be FeCl₂, NiCl₂, NiBr₂, NiI₂, (DME)NiBr₂, (DME)NiCl₂, PdCl₂, PdBr₂, Pd(OAc)₂, Pd(OTf)₂ or (COD)PdMeCl.

One embodiment of the present disclosure provides a preparation method of the Formula (II) complex comprising subjecting the Formula (I) compound to react with a nickel halide or its derivative at room temperature to obtain the Formula (II) complex.

In one embodiment of the present disclosure, the time of the reaction between the Formula (I) compound and the nickel halide or its coordination compound is 6 h to 24 h, and the nickel halide coordination compound may be (DME)NiBr₂.

In one embodiment of the present disclosure, after the reaction between the Formula (I) compound and the nickel halide or its coordination compound is completed, the product obtained is filtered, washed and dried.

One embodiment of the present disclosure provides a catalyst composition comprising a main catalyst and a cocatalyst, which can be used in the olefin polymerization, wherein the main catalyst is the Formula (II) complex and the cocatalyst may be at least one of alkyl aluminoxane, aluminum alkyl and halogenated aluminum alkyl.

For example, the cocatalyst may be one or a combination of methylaluminoxane (MAO), ethylaluminoxane (EAO), trimethylaluminium, triethylaluminum, triisobutylaluminium, tri-n-butyl aluminium, tri-n-hexyl aluminium, tri-n-pentylaluminium, tri-n-octylaluminium, diethylaluminium chloride, ethyl aluminum dichloride and ethylaluminum sesquichloride.

In one embodiment of the present disclosure, the molar ratio of the Formula (II) complex to the cocatalyst is 1:100 to 1:2000, for example, 1:200, 1:500, 1:800, 1:1000, 1:1200, 1:1500 and 1:1700.

In one embodiment of the present disclosure, the diketone, precursor of the diimine structure of the Formula (II) complex, is asymmetrical, but the substituted aniline moieties are symmetrical (the two substituted anilines have the same structure). On the one hand, the asymmetrical structure of the diketone can control the steric hindrance cooperated with the substituted aniline moieties and on the other hand, the asymmetrical conjugate ring system can regulate the electron effect transported to the central metal. Therefore, the Formula (II) complex has a high catalytic effect and can be used to prepare a highly branched polymer with a high activity.

One embodiment of the present disclosure provides a method for olefin polymerization using the catalyst composition above mentioned as catalytic system.

In one embodiment of the present disclosure, the alkene used in the polymerization may be one or a combination of ethylene, propylene, C₄-C₁₈ terminal olefin, C₄-C₁₈ nonterminal olefin and C₄-C₁₈ diolefin.

In one embodiment of the present disclosure, the reaction temperature of the olefin polymerization may be −78° C. to 200° C., preferably −20° C. to 150° C., more preferably 30° C. to 120° C.; and the reaction pressure may be 0.01 MPa to 10 MPa, preferably 0.01 MPa to 2 MPa.

In one embodiment of the present disclosure, the olefin polymerization is carried out in a solvent which may be an alkane, aromatic hydrocarbon or halohydrocarbon, for example, n-pentane, n-hexane, n-heptane, benzene, toluene, dichloroethane, dichloromethane or chloroform.

The catalyst composition formed by the Formula (II) complex and a cocatalyst in one embodiment of the present disclosure, has a high activity and can be used to catalyze the ethylene polymerization at high temperature to obtain a highly branched, controllable and low molecular weight oil-like polyolefin.

One embodiment of the present disclosure provides a preparation method of an alkane comprising steps of:

(a) using the Formula (II) complex as the main catalyst and an aluminum alkyl or halogenated aluminum alkyl as the cocatalyst to catalyze the olefin polymerization to obtain an oil-like polyolefin;

(b) subjecting the oil-like polyolefin obtained from the step (a) to a hydrogenation reaction to obtain a hydrogenated oil-like alkane.

In one embodiment of the present disclosure, the oil-like alkane obtained can be used as lubricant base oil, lubricant additive, rubber filling oil or resin processing aid.

Hereinafter, the preparation method of the catalyst composition and its application of an embodiment of the present disclosure will be further described by way of examples. The reagents used are all commercially available and the test methods are as follows:

NMR: a nuclear magnetic resonance spectrometer of Bruker (400 MHz) was used, CDCl₃ was used as the deuterated solvent and TMS was used as the internal standard sub stance.

Elemental Analysis: CHNS elemental analyzer of elementar Vario EL of Germany was used.

Catalytic Activity: gravimetric method was used. Catalytic Activity=Polymer Weight/(Reaction Time×Moles of the Catalyst Central Metal).

Polymer Molecular Weight: PL-GPC220 high temperature gel permeation chromatograph was used, 1,2,4-trichlorobenzene acted as mobile phase and the test temperature was 150° C.

Bromine Number: tested according to the standard with a number of SH/T 0236-1992 (2004).

Branching Degree: tested by HNMR.

### Example 1-1 Synthesis of Naphthofuran Diketone T1

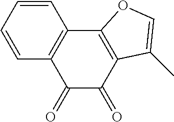 [SMILES: Cc1coc2c1C(=O)C(=O)c1ccccc1-2]

2 mmol of 2-hydroxy-1,4-naphoquinone, 10 mmol of bromopropanone, 10 mmol of NH₄OAc and 50 mL of dry toluene were added to a 100 mL stainless steel vessel, sealed, heated to 120° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and concentrated to give a dark red solid (339.2 mg) with a yield of 80%. The NMR data of the product were as follows. ¹H NMR (400 MHz, CDCl₃, δ, ppm): 7.78-7.98 (m, 3H), 7.48 (s, 1H), 7.36 (t, 1H), 2.01 (s, 3H).

### Example 1-2 Synthesis of Naphthofuran Diketone T2

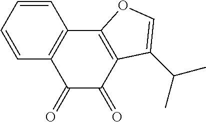 [SMILES: CC(C)c1coc2c1C(=O)C(=O)c1ccccc1-2]

2 mmol of 2-hydroxy-1,4-naphoquinone, 15 mmol of 1-bromo-3-methyl-2-butanone, 10 mmol of NH₄OAc and 50 mL of petroleum ether were added to a 100 mL stainless steel vessel, sealed, heated to 140° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and purified on a silica gel column with a mixed solvent of petroleum ether and ethyl acetate (50:1, v/v). The fractions were tested by silica gel plates and the second fraction was collected removing the solvent to give a dark red solid (408 mg) with a yield of 85%. The NMR data of the product were as follows. ¹H NMR (400 MHz, CDCl₃, δ, ppm): 7.78-7.98 (m, 3H), 7.48 (s, 1H), 7.36 (t, 1H), 2.28 (m, 1H), 1.18 (d, 6H).

### Example 1-3 Synthesis of Naphthofuran Diketone T3

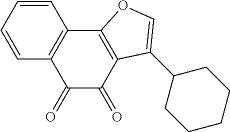 [SMILES: O=C1C(=O)c2c(C3CCCCC3)coc2-c2ccccc21]

2 mmol of 2-hydroxy-1,4-naphoquinone, 8 mmol of 2-bromo-1-cyclohexylethanone, 10 mmol of triethylamine and 50 mL of cyclohexane were added to a 100 mL stainless steel vessel, sealed, heated to 150° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and purified on a silica gel column with a mixed solvent of petroleum ether and ethyl acetate (50:1, v/v). The fractions were tested by silica gel plates and the second fraction was collected removing the solvent to give a dark red solid (498 mg) with a yield of 89%. The NMR data of the product were as follows. ¹H NMR (400 MHz, CDCl₃, δ, ppm): 7.78-7.96 (m, 3H), 7.48 (s, 1H), 7.36 (t, 1H), 2.72 (m, 1H), 1.61-1.86 (m, 4H), 1.43-1.53 (m, 6H).

### Example 1-4 Synthesis of Naphthofuran Diketone T4

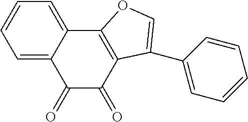 [SMILES: O=C1C(=O)c2c(-c3ccccc3)coc2-c2ccccc21]

2 mmol of 2-hydroxy-1,4-naphoquinone, 18 mmol of 2-bromoacetophenone, 10 mmol of NH₄OAc and 50 mL of dry toluene were added to a 100 mL stainless steel vessel, sealed, heated to 95° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and purified on a silica gel column with a mixed solvent of petroleum ether and ethyl acetate (50:1, v/v). The fractions were tested by silica gel plates and the second fraction was collected removing the solvent to give a dark red solid (493 mg) with a yield of 90%. The NMR data of the product were as follows. ¹H NMR (400 MHz, CDCl₃, δ, ppm): 7.78-7.96 (m, 3H), 7.75 (s, 1H), 7.41-7.51 (m, 5H), 7.36 (t, 1H).

### Example 1-5 Synthesis of Naphthofuran Diketone T5

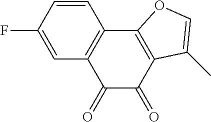 [SMILES: Cc1coc2c1C(=O)C(=O)c1cc(F)ccc1-2]

2 mmol of 2-hydroxy-7-fluoro-1,4-naphoquinone, 12 mmol of bromopropanone, 10 mmol of pyridine and 50 mL of dry toluene were added to a 100 mL stainless steel vessel, sealed, heated to 100° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and concentrated to give a dark red solid (368 mg) with a yield of 80%. The NMR data of the product were as follows. ¹H NMR (400 MHz, CDCl₃, δ, ppm): 7.59-7.94 (m, 3H), 7.48 (s, 1H), 2.01 (s, 1H).

### Example 1-6 Synthesis of Naphthofuran Diketone T6

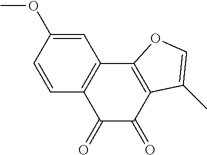

2 mmol of 2-hydroxy-6-methoxy-1,4-naphoquinone, 10 mmol of bromopropanone, 10 mmol of NH₄OAc and 60 mL of dry toluene were added to a 100 mL stainless steel vessel, sealed, heated to 120° C. and reacted for 10 h. After the reaction was completed, the resulting mixture was cooled to room temperature, acidized with 30 mL hydrochloric acid (1 mol/L) and then extracted with ethyl acetate (200 mL×3). The ethyl acetate layers were collected, washed with water, dried with anhydrous sodium sulfate, filtered and concentrated to give a dark red solid (368 mg) with a yield of 80%. The NMR data of the product were as follows. ¹H NMR (400 MHz, CDCl₃, δ, ppm): 8.04 (d, 1H), 7.48 (s, 2H), 7.22 (d, 2H), 3.81 (s, 3H), 2.01 (s, 3H).

### Example 2-1 Synthesis of Naphthofuran Diimine L11

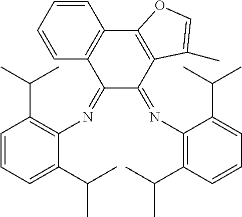 [SMILES: Cc1coc2c1C(=N\c1c(C(C)C)cccc1C(C)C)/C(=N/c1c(C(C)C)cccc1C(C)C)c1ccccc1-2]

The compound T1 (4 mmol, 0.848 g) and 2,6-diisopropylaniline (9 mmol, 1.594 g) used as reactants, and p-toluenesulfonic acid (40 mg) used as a catalyst, were refluxed with 100 mL of toluene for 1 day at 100° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 60%. The NMR data of the product were as follows. ¹H NMR (400 MHz, CDCl₃, δ, ppm): 7.60-8.00 (m, 4H), 7.55 (s, 1H), 7.48 (t, 2H), 7.22 (d, 4H), 2.88 (m, 4H), 2.01 (s, 3H), 1.18 (d, 24H).

### Example 2-2 Synthesis of Naphthofuran Diimine L12

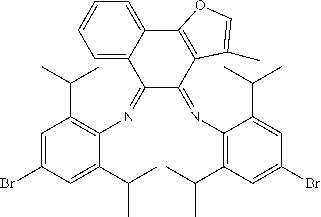 [SMILES: Cc1coc2c1C(=N\c1c(C(C)C)cc(Br)cc1C(C)C)/C(=N/c1c(C(C)C)cc(Br)cc1C(C)C)c1ccccc1-2]

The compound T1 (4 mmol, 0.848 g) and 4-bromo-2,6-diisopropylaniline (12 mmol, 3.061 g) used as reactants, and p-toluenesulfonic acid (50 mg) used as a catalyst, were refluxed with 100 mL of toluene for 36 h at 110° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 63%. The NMR data of the product were as follows. ¹H NMR (400 MHz, CDCl₃, δ, ppm): 7.60-8.00 (m, 4H), 7.55 (s, 1H), 7.38 (s, 4H), 2.88 (m, 4H), 2.01 (s, 3H), 1.18 (d, 24H).

### Example 2-3 Synthesis of Naphthofuran Diimine L13

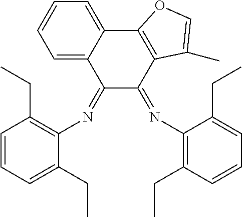 [SMILES: CCc1cccc(CC)c1/N=C1/C(=N/c2c(CC)cccc2CC)c2c(C)coc2-c2ccccc21]

The compound T1 (4 mmol, 0.848 g) and 2,6-diethylaniline (10 mmol, 1.491 g) used as reactants, and trifluoromethanesulfonic acid (48 mg) used as a catalyst, were refluxed with 100 mL of toluene for 1 day at 100° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 67%. The NMR data of the product were as follows. ¹H NMR (400 MHz, CDCl₃, δ, ppm): 7.60-8.00 (m, 4H), 7.55 (s, 1H), 7.48 (t, 2H), 7.10 (d, 4H), 2.71 (q, 8H), 2.01 (s, 3H), 1.18 (t, 12H).

### Example 2-4 Synthesis of Naphthofuran Diimine L14

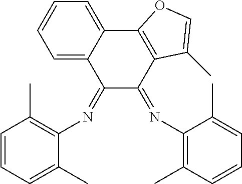 [SMILES: Cc1cccc(C)c1/N=C1/C(=N/c2c(C)cccc2C)c2c(C)coc2-c2ccccc21]

The compound T1 (4 mmol, 0.848 g) and 2,6-dimethylaniline (9 mmol, 1.090 g) used as reactants, and trifluoromethanesulfonic acid (50 mg) used as a catalyst, were refluxed with 100 mL of toluene for 1 day at 110° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 71%. The NMR data of the product were as follows. ¹H NMR (400 MHz, CDCl₃, δ, ppm): 7.60-8.00 (m, 4H), 7.55 (s, 1H), 7.24 (m, 6H), 2.55 (s, 12H), 2.01 (s, 3H).

### Example 2-5 Synthesis of Naphthofuran Diimine L21

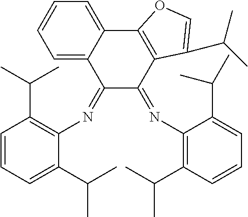 [SMILES: CC(C)c1cccc(C(C)C)c1/N=C1/C(=N/c2c(C(C)C)cccc2C(C)C)c2c(C(C)C)coc2-c2ccccc21]

The compound T2 (4 mmol, 0.960 g) and 2,6-diisopropylaniline (9 mmol, 1.594 g) used as reactants, and p-toluenesulfonic acid (103 mg) used as a catalyst, were refluxed with 100 mL of ethanol for 2 days at 110° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 55%. The NMR data of the product were as follows. ¹H NMR (400 MHz, CDCl₃, δ, ppm): 7.60-8.00 (m, 4H), 7.55 (s, 1H), 7.48 (t, 2H), 7.22 (d, 4H), 2.88 (m, 5H), 1.88 (d, 30H).

### Example 2-6 Synthesis of Naphthofuran Diimine L22

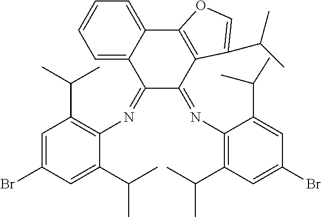 [SMILES: CC(C)c1cc(Br)cc(C(C)C)c1/N=C1/C(=N/c2c(C(C)C)cc(Br)cc2C(C)C)c2c(C(C)C)coc2-c2ccccc21]

The compound T2 (4 mmol, 0.960 g) and 4-bromo-2,6-diisopropylaniline (12 mmol, 3.061 g) used as reactants, and p-toluenesulfonic acid (70 mg) used as a catalyst, were refluxed with 100 mL of toluene for 2 days at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 56%. The NMR data of the product were as follows. ¹H NMR (400 MHz, CDCl₃, δ, ppm): 7.60-8.00 (m, 4H), 7.55 (s, 1H), 7.38 (s, 4H), 2.88 (m, 5H), 1.18 (d, 30H).

### Example 2-7 Synthesis of Naphthofuran Diimine L23

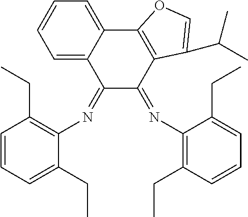 [SMILES: CCc1cccc(CC)c1/N=C1/C(=N/c2c(CC)cccc2CC)c2c(C(C)C)coc2-c2ccccc21]

The compound T2 (4 mmol, 0.960 g) and 2,6-diethylaniline (15 mmol, 2.237 g) used as reactants, and p-toluenesulfonic acid (80 mg) used as a catalyst, were refluxed with 100 mL of toluene for 50 h at 100° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 60%. The NMR data of the product were as follows. ¹H NMR (400 MHz, CDCl₃, δ, ppm): 7.60-8.00 (m, 4H), 7.55 (s, 1H), 7.48 (t, 2H), 7.10 (d, 4H), 2.88 (d, 1H), 2.71 (q, 8H), 1.18 (m, 18H).

### Example 2-8 Synthesis of Naphthofuran Diimine L24

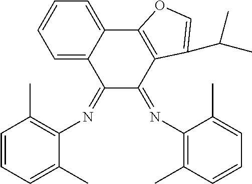 [SMILES: Cc1cccc(C)c1/N=C1/C(=N/c2c(C)cccc2C)c2c(C(C)C)coc2-c2ccccc21]

The compound T2 (4 mmol, 0.960 g) and 2,6-dimethylaniline (15 mmol, 1.816 g) used as reactants, and trifluoromethanesulfonic acid (70 mg) used as a catalyst, were refluxed with 100 mL of ethanol for 90 h at 100° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 63%. The NMR data of the product were as follows. ¹H NMR (400 MHz, CDCl₃, δ, ppm): 7.60-8.00 (m, 4H), 7.55 (s, 1H), 7.24 (m, 6H), 2.88 (m, 1H), 2.55 (s, 12H), 1.18 (d, 6H).

### Example 2-9 Synthesis of Naphthofuran Diimine L31

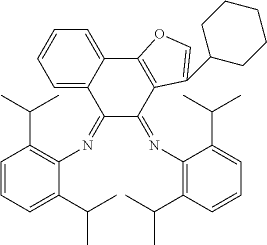 [SMILES: CC(C)c1cccc(C(C)C)c1/N=C1/C(=N/c2c(C(C)C)cccc2C(C)C)c2c(C3CCCCC3)coc2-c2ccccc21]

The compound T3 (4 mmol, 1.120 g) and 2,6-diisopropylaniline (20 mmol, 3.543 g) used as reactants, and p-toluenesulfonic acid (80 mg) used as a catalyst, were refluxed with 100 mL of toluene for 72 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 51%. The NMR data of the product were as follows. ¹H NMR (400 MHz, CDCl₃, δ, ppm): 7.60-8.00 (m, 4H), 7.55 (s, 1H), 7.48 (t, 2H), 7.22 (d, 4H), 2.88 (m, 4H), 2.72 (m, 1H), 1.43-1.86 (m, 10H), 1.18 (d, 24H).

### Example 2-10 Synthesis of Naphthofuran Diimine L32

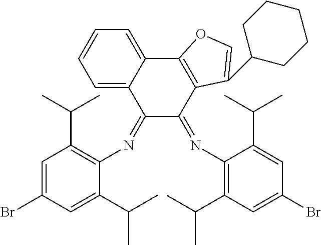 [SMILES: CC(C)c1cc(Br)cc(C(C)C)c1/N=C1/C(=N/c2c(C(C)C)cc(Br)cc2C(C)C)c2c(C3CCCCC3)coc2-c2ccccc21]

The compound T3 (4 mmol, 1.120 g) and 4-bromo-2,6-diisopropylaniline (15 mmol, 3.826 g) used as reactants, and trifluoromethanesulfonic acid (90 mg) used as a catalyst, were refluxed with 100 mL of toluene for 72 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 56%. The NMR data of the product were as follows. ¹H NMR (400 MHz, CDCl₃, δ, ppm): 7.60-8.00 (m, 4H), 7.55 (s, 1H), 7.38 (s, 4H), 2.88 (m, 4H), 2.72 (m, 1H), 1.43-1.86 (m, 10H), 1.18 (d, 24H).

### Example 2-11 Synthesis of Naphthofuran Diimine L33

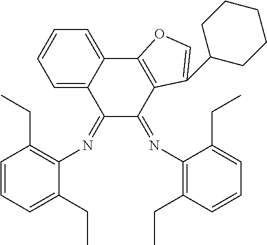 [SMILES: CCc1cccc(CC)c1/N=C1/C(=N/c2c(CC)cccc2CC)c2c(C3CCCCC3)coc2-c2ccccc21]

The compound T3 (4 mmol, 1.120 g) and 2,6-diethylaniline (15 mmol, 2.237 g) used as reactants, and p-toluenesulfonic acid (90 mg) used as a catalyst, were refluxed with 100 mL of toluene for 72 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 58%. The NMR data of the product were as follows. ¹H NMR (400 MHz, CDCl₃, δ, ppm): 7.60-8.00 (m, 4H), 7.55 (s, 1H), 7.48 (t, 2H), 7.10 (d, 4H), 2.71 (m, 9H), 1.43-1.86 (m, 10H), 1.18 (t, 12H).

### Example 2-12 Synthesis of Naphthofuran Diimine L34

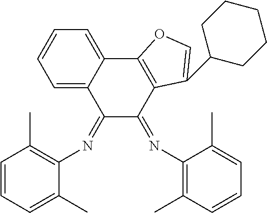 [SMILES: Cc1cccc(C)c1/N=C1/C(=N/c2c(C)cccc2C)c2c(C3CCCCC3)coc2-c2ccccc21]

The compound T3 (4 mmol, 1.120 g) and 2,6-dimethylaniline (15 mmol, 1.816 g) used as reactants, and p-toluenesulfonic acid (60 mg) used as a catalyst, were refluxed with 100 mL of toluene for 72 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 65%. The NMR data of the product were as follows. ¹H NMR (400 MHz, CDCl₃, δ, ppm): 7.60-8.00 (m, 4H), 7.55 (s, 1H), 7.24 (m, 6H), 2.55 (s, 12H), 2.72 (m, 1H), 1.43-1.86 (m, 10H).

### Example 2-13 Synthesis of Naphthofuran Diimine L41

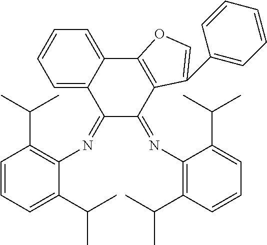 [SMILES: CC(C)c1cccc(C(C)C)c1/N=C1/C(=N/c2c(C(C)C)cccc2C(C)C)c2c(-c3ccccc3)coc2-c2ccccc21]

The compound T4 (4 mmol, 1.104 g) and 2,6-diisopropylaniline (20 mmol, 3.543 g) used as reactants, and p-toluenesulfonic acid (60 mg) used as a catalyst, were refluxed with 100 mL of toluene for 48 h at 110° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 50%. The NMR data of the product were as follows. ¹H NMR (400 MHz, CDCl₃, δ, ppm): 7.60-8.00 (m, 4H), 7.82 (s, 1H), 7.41-7.51 (m, 7H), 7.22 (d, 4H), 2.88 (m, 4H), 1.18 (d, 24H).

### Example 2-14 Synthesis of Naphthofuran Diimine L42

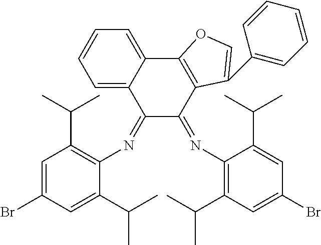 [SMILES: CC(C)c1cc(Br)cc(C(C)C)c1/N=C1/C(=N/c2c(C(C)C)cc(Br)cc2C(C)C)c2c(-c3ccccc3)coc2-c2ccccc21]

The compound T4 (4 mmol, 1.104 g) and 4-bromo-2,6-diisopropylaniline (18 mmol, 4.591 g) used as reactants, and p-toluenesulfonic acid (70 mg) used as a catalyst, were refluxed with 100 mL of toluene for 48 h at 100° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 53%. The NMR data of the product were as follows. ¹H NMR (400 MHz, CDCl₃, δ, ppm): 7.60-8.00 (m, 4H), 7.82 (s, 1H), 7.41-7.51 (m, 9H), 2.88 (m, 4H), 1.18 (d, 24H).

### Example 2-15 Synthesis of Naphthofuran Diimine L43

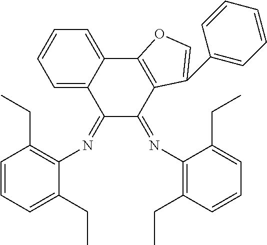 [SMILES: CCc1cccc(CC)c1/N=C1/C(=N/c2c(CC)cccc2CC)c2c(-c3ccccc3)coc2-c2ccccc21]

The compound T4 (4 mmol, 1.104 g) and 2,6-diethylaniline (15 mmol, 2.239 g) used as reactants, and p-toluenesulfonic acid (80 mg) used as a catalyst, were refluxed with 100 mL of toluene for 36 h at 110° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 57%. The NMR data of the product were as follows. ¹H NMR (400 MHz, CDCl₃, δ, ppm): 7.60-8.00 (m, 4H), 7.82 (s, 1H), 7.41-7.51 (m, 7H), 7.10 (d, 4H), 2.71 (q, 8H), 1.18 (t, 12H).

### Example 2-16 Synthesis of Naphthofuran Diimine L44

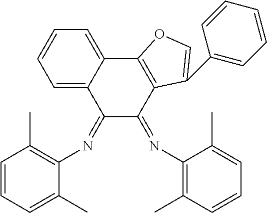 [SMILES: Cc1cccc(C)c1/N=C1/C(=N/c2c(C)cccc2C)c2c(-c3ccccc3)coc2-c2ccccc21]

The compound T4 (4 mmol, 1.104 g) and 2,6-dimethylaniline (15 mmol, 1.816 g) used as reactants, and p-toluenesulfonic acid (50 mg) used as a catalyst, were refluxed with 100 mL of toluene for 48 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 63%. The NMR data of the product were as follows. ¹H NMR (400 MHz, CDCl₃, δ, ppm): 7.60-8.00 (m, 4H), 7.82 (s, 1H), 7.41-7.51 (m, 5H), 7.24 (m, 6H), 2.55 (s, 12H).

### Example 2-17 Synthesis of Naphthofuran Diimine L55

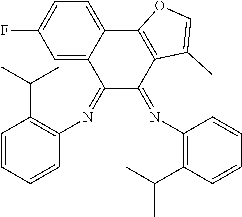 [SMILES: Cc1coc2c1C(=N\c1ccccc1C(C)C)/C(=N/c1ccccc1C(C)C)c1cc(F)ccc1-2]

The compound T5 (4 mmol, 0.920 g) and 2-isopropylaniline (10 mmol, 1.351 g) used as reactants, and p-toluenesulfonic acid (50 mg) used as a catalyst, were refluxed with 100 mL of toluene for 48 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 63%. The NMR data of the product were as follows. ¹H NMR (400 MHz, CDCl₃, δ, ppm): 7.83-7.99 (m, 2H), 7.55 (t, 2H), 7.06-7.32 (m, 8H), 2.88 (s, 1H), 2.01 (s, 3H), 1.18 (d, 6H).

### Example 2-18 Synthesis of Naphthofuran Diimine L66

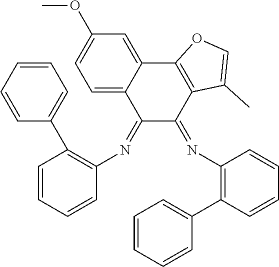 [SMILES: COc1ccc2c(c1)-c1occ(C)c1C(=N\c1ccccc1-c1ccccc1)/C2=N/c1ccccc1-c1ccccc1]

The compound T6 (4 mmol, 0.968 g) and 2-aminodiphenyl (16 mmol, 2.705 g) used as reactants, and p-toluenesulfonic acid (80 mg) used as a catalyst, were refluxed with 100 mL of toluene for 72 h at 120° C. After the reaction was completed, the solvent was removed and the residue was purified on a silica gel column with a mixed solvent of petroleum ether and ethyl acetate (50:1, v/v). The fractions were tested by silica gel plates and the second fraction was collected removing the solvent to give a yellow solid with a yield of 63%. The NMR data of the product were as follows. ¹H NMR (400 MHz, CDCl₃, δ, ppm): 8.00 (d, 1H), 7.77 (d, 2H), 7.55 (s, 1H), 7.15-7.51 (m, 18H), 3.81 (s, 3H), 2.01 (s, 3H).

### Example 3-1 Synthesis of Naphthofuran Diimine Complex C11

Under the protection of inert gas, 0.2 mmol of (DME)NiBr₂ dissolved in dichloromethane was added dropwise to a solution of the compound L11 in dichloromethane at room temperature, stirred for 8 h and added with n-hexane to precipitate a red solid. The resulting mixture was filtered, washed with hexane and dried to give the red solid C11 with a yield of 89%. The elemental analysis data (%) of the product (C₃₇H₄₂Br₂N₂NiO) were as follows: C, 59.51; H, 5.60; N, 3.84; 0, 2.19.

### Example 3-2 Synthesis of Naphthofuran Diimine Complex C12

Procedures similar to the Example 3-1 were conducted with a difference of using the compound L12 as the diimine to obtain a red solid C12 having a yield of 90%. The elemental analysis data (%) of the product (C₂₇H₄₀Br₄N₂NiO) were as follows: C, 49.05; H, 4.50; N, 3.03; 0, 1.83.

### Example 3-3 Synthesis of Naphthofuran Diimine Complex C13

Procedures similar to the Example 3-1 were conducted with a difference of using the compound L13 as the diimine to obtain a red solid C12 having a yield of 85%. The elemental analysis data (%) of the product (C₃₃H₃₄Br₂N₂NiO) were as follows: C, 57.10; H, 4.88; N, 4.14; 0, 2.38.

### Example 3-4 Synthesis of Naphthofuran Diimine Complex C14

Procedures similar to the Example 3-1 were conducted with a difference of using the compound L14 as the diimine to obtain a red solid C14 having a yield of 88%. The elemental analysis data (%) of the product (C₂₉H₂₆Br₂N₂NiO) were as follows: C, 54.72; H, 4.23; N, 4.36; 0, 2.56.

### Example 3-5 Synthesis of Naphthofuran Diimine Complex C21

Procedures similar to the Example 3-1 were conducted with a difference of using the compound L21 as the diimine to obtain a red solid C21 having a yield of 84%. The elemental analysis data (%) of the product (C₃₉H₄₆Br₂N₂NiO) were as follows: C, 60.38; H, 6.03; N, 3.56; 0, 2.07.

### Example 3-6 Synthesis of Naphthofuran Diimine Complex C22

Procedures similar to the Example 3-1 were conducted with a difference of using the compound L22 as the diimine to obtain a red solid C22 having a yield of 86%. The elemental analysis data (%) of the product (C₃₉H₄₄Br₄N₂NiO) were as follows: C, 50.13; H, 4.85; N, 3.03; 0, 1.76.

### Example 3-7 Synthesis of Naphthofuran Diimine Complex C23

Procedures similar to the Example 3-1 were conducted with a difference of using the compound L23 as the diimine to obtain a red solid C23 having a yield of 88%. The elemental analysis data (%) of the product (C₃₅H₃₈Br₂N₂NiO) were as follows: C, 58.35; H, 5.36; N, 3.95; 0, 2.26.

### Example 3-8 Synthesis of Naphthofuran Diimine Complex C24

Procedures similar to the Example 3-1 were conducted with a difference of using the compound L24 as the diimine to obtain a red solid C24 having a yield of 91%. The elemental analysis data (%) of the product (C₃₁₁-130Br₂N₂NiO) were as follows: C, 55.92; H, 4.53; N, 4.13; 0, 2.43.

### Example 3-9 Synthesis of Naphthofuran Diimine Complex C31

Procedures similar to the Example 3-1 were conducted with a difference of using the compound L31 as the diimine to obtain a red solid C31 having a yield of 90%. The elemental analysis data (%) of the product (C₄₂H₅₀Br₂N₂NiO) were as follows: C, 61.79; H, 6.23; N, 3.58; 0, 2.03.

### Example 3-10 Synthesis of Naphthofuran Diimine Complex C32

Procedures similar to the Example 3-1 were conducted with a difference of using the compound L32 as the diimine to obtain a red solid C32 having a yield of 86%. The elemental analysis data (%) of the product (C₄₂H₄₈Br₄N₂NiO) were as follows: C, 51.79; H, 4.92; N, 2.96; 0, 1.60.

### Example 3-11 Synthesis of Naphthofuran Diimine Complex C33

Procedures similar to the Example 3-1 were conducted with a difference of using the compound L33 as the diimine to obtain a red solid C33 having a yield of 84%. The elemental analysis data (%) of the product (C₃₈H₄₂Br₂N₂NiO) were as follows: C, 60.12; H, 5.58; N, 3.75; 0, 2.06.

### Example 3-12 Synthesis of Naphthofuran Diimine Complex C34

Procedures similar to the Example 3-1 were conducted with a difference of using the compound L34 as the diimine to obtain a red solid C34 having a yield of 89%. The elemental analysis data (%) of the product (C₃₄H₃₄Br₂N₂NiO) were as follows: C, 57.98; H, 4.92; N, 3.91; 0, 2.34.

### Example 3-13 Synthesis of Naphthofuran Diimine Complex C41

Procedures similar to the Example 3-1 were conducted with a difference of using the compound L41 as the diimine to obtain a red solid C41 having a yield of 89%. The elemental analysis data (%) of the product (C₄₂H₄₄Br₂N₂NiO) were as follows: C, 62.23; H, 5.52; N, 3.56; 0, 2.06.

### Example 3-14 Synthesis of Naphthofuran Diimine Complex C42

Procedures similar to the Example 3-1 were conducted with a difference of using the compound L42 as the diimine to obtain a red solid C42 having a yield of 92%. The elemental analysis data (%) of the product (C₄₂H₄₂Br₄N₂NiO) were as follows: C, 52.15; H, 4.40; N, 2.95; 0, 1.71.

### Example 3-15 Synthesis of Naphthofuran Diimine Complex C43

Procedures similar to the Example 3-1 were conducted with a difference of using the compound L43 as the diimine to obtain a red solid C43 having a yield of 90%. The elemental analysis data (%) of the product (C₃₈₁-136Br₂N₂NiO) were as follows: C, 60.50; H, 4.85; N, 3.78; 0, 2.18.

### Example 3-16 Synthesis of Naphthofuran Diimine Complex C44

Procedures similar to the Example 3-1 were conducted with a difference of using the compound L44 as the diimine to obtain a red solid C44 having a yield of 91%. The elemental analysis data (%) of the product (C₃₄H₂₈Br₂NNiO) were as follows: C, 58.50; H, 4.14; N, 3.95; 0, 2.36.

### Example 3-17 Synthesis of Naphthofuran Diimine Complex C55

Under the protection of inert gas, 0.2 mmol of (DME)NiBr₂ dissolved in dichloromethane was added dropwise to a solution of the compound L55 in dichloromethane at room temperature, stirred for 8 h and added with n-hexane to precipitate a red solid. The resulting mixture was filtered, washed with hexane and dried to give the red solid C55 with a yield of 82%. The elemental analysis data (%) of the product (C₃₁H₂₉Br₂N₂FNiO) were as follows: C, 54.50; H, 4.18; N, 4.15; 0, 2.35.

### Example 3-18 Synthesis of Naphthofuran Diimine Complex C66

Under the protection of inert gas, 0.2 mmol of (DME)NiBr₂ dissolved in dichloromethane was added dropwise to a solution of the compound L66 in dichloromethane at room temperature, stirred for 8 h and added with n-hexane to precipitate a red solid. The resulting mixture was filtered, washed with hexane and dried to give the red solid C66 with a yield of 85%. The elemental analysis data (%) of the product (C₃₈H₂₈Br₂N₂NiO₂) were as follows: C, 59.85; H, 3.90; N, 3.63; 0, 4.12.

### Example 3-19 Synthesis of Naphthofuran Diimine Complex C55Pd

Under the protection of inert gas, 0.2 mmol of (COD)PdMeCl dissolved in dichloromethane was added dropwise to a solution of the compound L55 in dichloromethane at room temperature, stirred for 12 h, concentrated under vacuum and then added with n-hexane to precipitate a red solid. The resulting mixture was filtered, washed with hexane and dried to give the red solid C55Pd with a yield of 85%. The elemental analysis data (%) of the product (C₃₂H₃₂ClFN₂PdO) were as follows: C, 61.88; H, 5.13; N, 4.56; 0, 2.55.

### Example 3-20 Synthesis of Naphthofuran Diimine Complex C55Fe

Under the protection of inert gas, 0.5 mmol of FeCl₂.4H₂O dissolved in 20 mL of tetrahydrofuran was added to a solution of the compound L55 in dichloromethane at room temperature, stirred for 36 h, concentrated under vacuum and added with n-hexane to precipitate a red solid. The resulting mixture was filtered, washed with hexane and dried to give a blue solid C55Fe with a yield of 81%. The elemental analysis data (%) of the product (C₃₁H₂₉C₁₂FN₂FeO) were as follows: C, 62.90; H, 4.96; N, 4.78; 0, 2.68.

### Example 3-21 Synthesis of Naphthofuran Diimine Complex C55NiCl

Under the protection of inert gas, 0.2 mmol of (DME)NiCl₂ dissolved in dichloromethane was added dropwise to a solution of the compound L55 in dichloromethane at room temperature, stirred for 16 h, concentrated under vacuum and then added with n-hexane to precipitate a red solid. The resulting mixture was filtered, washed with hexane and dried to give the red solid C55NiCl with a yield of 75%. The elemental analysis data (%) of the product (C₃₁H₂₉C₁₂FN₂NiO) were as follows: C, 62.63; H, 4.95; N, 4.68; 0, 2.65.

### Application Example 1 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C11 (1 μmol) was added at 30° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 2.2×10⁶ g/(mol_{Ni}.h) and the oil-like polyethylene had a branching degree of 123 CH₃/1000 C.

### Application Example 2 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C11 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.6×10⁶ g/(mol_{Ni}.h) and the oil-like polyethylene had a branching degree of 136 CH₃/1000 C.

### Application Example 3 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C11 (1 μmol) was added at 70° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 3.4×10⁶ g/(mol_{Ni}.h) and the oil-like polyethylene had a branching degree of 162 CH₃/1000 C.

### Application Example 4 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of MAO as a cocatalyst in toluene were added, the complex C11 (1 μmol) was added at 60° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.3×10⁶ g/(mol_{Ni}.h) and the oil-like polyethylene had a branching degree of 125 CH₃/1000 C.

### Application Example 5 Ethylene Polymerization

A 250 mL polymerization vial was filled with ethylene after underwent 3 cycles of vacuum and filling with N₂. Under ethylene atmosphere, 50 mL of toluene as a solvent and a solution (0.5 mL, 1 mol/L) of MMAO (modified methylaluminoxane) as a cocatalyst in toluene were added, the complex C11 (1 μmol) was added at 60° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.8×10⁶ g/(mol_{Ni}.h) and the oil-like polyethylene had a branching degree of 134 CH₃/1000 C.

### Application Example 6 Ethylene and Propylene Polymerization

A 250 mL polymerization vial was filled with gases of ethylene and propylene (regulated to V(ethylene):V(propylene)=3:1 by flowmeters) after underwent 3 cycles of vacuum and filling with N₂. Under the alkenes atmosphere, 50 mL of toluene as a solvent and a solution (1 mL, 1 mol/L) of diethylaluminium chloride as a cocatalyst in toluene were added, the complex C11 (1 μmol) was added at 60° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.6×10⁶ g/(mol_{Ni}.h) and the oil-like polymer had a branching degree of 262 CH₃/1000 C.

### Application Example 7 Ethylene and Hexene Polymerization

A 250 mL polymerization vial was filled with ethylene after underwent 3 cycles of vacuum and filling with N₂. Under ethylene atmosphere, 50 mL of toluene as a solvent, 2 mL of 1-hexene and a solution (1 mL, 1 mol/L) of diethylaluminium chloride as a cocatalyst in toluene were added, the complex C11 (1 μmol) was added at 60° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.6×10⁶ g/(mol_{Ni}.h) and the oil-like polymer had a branching degree of 212 CH₃/1000 C.

### Application Example 8 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C12 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.6×10⁶ g/(mol_{Ni}.h) and the oil-like polyethylene had a branching degree of 152 CH₃/1000 C.

### Application Example 9 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C13 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.3×10⁶ g/(mol_{Ni}.h) and the oil-like polyethylene had a branching degree of 115 CH₃/1000 C.

### Application Example 10 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C14 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 6.8×10⁶ g/(mol_{Ni}.h) and the oil-like polyethylene had a branching degree of 86 CH₃/1000 C.

### Application Example 11 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C21 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 3.7×10⁶ g/(mol_{Ni}.h) and the oil-like polyethylene had a branching degree of 158 CH₃/1000 C.

### Application Example 12 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C22 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 3.2×10⁶ g/(mol_{Ni}.h) and the oil-like polyethylene had a branching degree of 186 CH₃/1000 C.

### Application Example 13 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C23 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.9×10⁶ g/(mol_{Ni}.h) and the oil-like polyethylene had a branching degree of 142 CH₃/1000 C.

### Application Example 14 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C24 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.7×10⁶ g/(mol_{Ni}.h) and the oil-like polyethylene had a branching degree of 110 CH₃/1000 C.

### Application Example 15 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C31 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.1×10⁶ g/(mol_{Ni}.h) and the oil-like polyethylene had a branching degree of 170 CH₃/1000 C.

### Application Example 16 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C32 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 4.5×10⁶ g/(mol_{Ni}.h) and the oil-like polyethylene had a branching degree of 192 CH₃/1000 C.

### Application Example 17 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C33 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.3×10⁶ g/(mol_{Ni}.h) and the oil-like polyethylene had a branching degree of 126 CH₃/1000 C.

### Application Example 18 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C34 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 7.5×10⁶ g/(mol_{Ni}.h) and the oil-like polyethylene had a branching degree of 73 CH₃/1000 C.

### Application Example 19 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C41 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 2.1×10⁶ g/(mol_{Ni}.h) and the oil-like polyethylene had a branching degree of 164 CH₃/1000 C.

### Application Example 20 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C42 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 1.8×10⁶ g/(mol_{Ni}.h) and the oil-like polyethylene had a branching degree of 171 CH₃/1000 C.

### Application Example 21 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C43 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 6.1×10⁶ g/(mol_{Ni}.h) and the oil-like polyethylene had a branching degree of 104 CH₃/1000 C.

### Application Example 22 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C44 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 8.4×10⁶ g/(mol_{Ni}.h) and the oil-like polyethylene had a branching degree of 96 CH₃/1000 C.

### Application Example 23 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C55 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 7.2×10⁶ g/(mol_{Ni}.h) and the oil-like polyethylene had a branching degree of 125 CH₃/1000 C.

### Application Example 24 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of MAO as a cocatalyst in toluene were added, the complex C55Fe (1 μmol) was added at 50° C. polymerizing for 30 min. The ethylene was cut off and 1 mL of methanolic hydrochloric acid solution (5%) was added to the toluene solution to quench the reaction. The reaction liquid was removed to give a waxy polymer. The catalytic activity was 5.2×10⁶ g/(mol_{Fe}.h).

### Application Example 25 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of MAO as a cocatalyst in toluene were added, the complex C55NiCl (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 3.4×10⁶ g/(mol_{Ni}.h) and the oil-like polyethylene had a branching degree of 89 CH₃/1000 C.

### Application Example 26 Ethylene Polymerization

A 250 mL polymerization vial was filled with ethylene after underwent 3 cycles of vacuum and filling with N₂. Under ethylene atmosphere, 50 mL of toluene as a solvent and a solution (1 mL, 1 mol/L) of diethylaluminium chloride as a cocatalyst in toluene were added, the complex C66 (1 μmol) was added at 50° C. polymerizing for 30 min. The ethylene was cut off and 1 mL of methanolic hydrochloric acid solution (5%) was added to the toluene solution to quench the reaction. The reaction liquid was removed to give an oil-like polymer. The catalytic activity was 5.6×10⁶ g/(mol_{Ni}.h) and the oil-like polyethylene had a branching degree of 112 CH₃/1000 C.

### Application Example 27 Ethylene Polymerization

A 250 mL polymerization vial was filled with ethylene after underwent 3 cycles of vacuum and filling with N₂. Under ethylene atmosphere, 50 mL of n-hexane as a solvent and a solution (1 mL, 1 mol/L) of diethylaluminium chloride as a cocatalyst in toluene were added, the complex C55 (1 μmol) was added at 50° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 7.6×10⁶ g/(mol_{Ni}.h) and the oil-like polyethylene had a branching degree of 142 CH₃/1000 C.

### Application Example 28 Ethylene Polymerization

A 250 mL polymerization vial was filled with ethylene after underwent 3 cycles of vacuum and filling with N₂. Under ethylene atmosphere, 50 mL of dichloroethane as a solvent and a solution (1 mL, 1 mol/L) of diethylaluminium chloride as a cocatalyst in toluene were added, the complex C55 (1 μmol) was added at 50° C. polymerizing for 30 min. The ethylene was cut off and 1 mL of methanolic hydrochloric acid solution (5%) was added to the dichloroethane solution to quench the reaction. The reaction liquid was removed to give an oil-like polymer. The catalytic activity was 6.2×10⁶ g/(mol_{Ni}.h) and the oil-like polyethylene had a branching degree of 121 CH₃/1000 C.

### Application Example 29 Ethylene Polymerization

A 250 mL polymerization vial was filled with ethylene after underwent 3 cycles of vacuum and filling with N₂. Under ethylene atmosphere, 50 mL of n-hexane as a solvent and a solution (1 mL, 1 mol/L) of diethylaluminium chloride as a cocatalyst in toluene were added, the complex C55 (1 μmol) was added at 60° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 8.5×10⁶ g/(mol_{Ni}.h) and the oil-like polyethylene had a branching degree of 151 CH₃/1000 C.

### Application Example 30 Ethylene Polymerization

A 250 mL polymerization vial was filled with ethylene after underwent 3 cycles of vacuum and filling with N₂. Under ethylene atmosphere, 50 mL of n-hexane as a solvent and a solution (0.5 mL, 1 mol/L) of diethylaluminium chloride as a cocatalyst in toluene were added, the complex C55 (1 μmol) was added at 60° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 5.9×10⁶ g/(mol_{Ni}.h) and the oil-like polyethylene had a branching degree of 156 CH₃/1000 C.

### Application Example 31 Ethylene Polymerization

A 250 mL polymerization vial was filled with ethylene after underwent 3 cycles of vacuum and filling with N₂. Under ethylene atmosphere, 50 mL of n-hexane as a solvent and a solution (2 mL, 1 mol/L) of diethylaluminium chloride as a cocatalyst in toluene were added, the complex C55 (1 μmol) was added at 60° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 9.3×10⁶ g/(mol_{Ni}.h) and the oil-like polyethylene had a branching degree of 120 CH₃/1000 C.

### Application Example 32 Ethylene Polymerization

A 250 mL polymerization vial was filled with ethylene after underwent 3 cycles of vacuum and filling with N₂. Under ethylene atmosphere, 50 mL of n-hexane as a solvent and a solution (1 mL, 1 mol/L) of diethylaluminium chloride as a cocatalyst in toluene were added, the complex C55 (1 μmol) was added at 70° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 8.0×10⁶ g/(mol_{Ni}.h) and the oil-like polyethylene had a branching degree of 182 CH₃/1000 C.

### Application Example 33 Ethylene Polymerization

A 250 mL polymerization vial was filled with ethylene after underwent 3 cycles of vacuum and filling with N₂. Under ethylene atmosphere, 50 mL of n-hexane as a solvent and a solution (1 mL, 1 mol/L) of diethylaluminium chloride as a cocatalyst in toluene were added, the complex C55 (1 μmol) was added at 90° C. polymerizing for 30 min. The ethylene was cut off and 1 mL of methanolic hydrochloric acid solution (5%) was added to the n-hexane solution to quench the reaction. The reaction liquid was removed to give an oil-like polymer. The catalytic activity was 6.4×10⁶ g/(mol_{Ni}.h) and the oil-like polyethylene had a branching degree of 232 CH₃/1000 C.

### Application Example 34 Ethylene Polymerization

A 2 L polymerizer was heated to 100° C., underwent 3 cycles of vacuum and filling with N₂ and cooled to 60° C. Under nitrogen atmosphere, 1.2 L of n-hexane as a solvent, a solution (20 mL, 1 mol/L) of diethylaluminium chloride as a cocatalyst in toluene and the complex C55 (25 μmol) were added polymerizing for 30 min under an ethylene pressure of 1 atm. The ethylene was cut off and 1 mL of methanolic hydrochloric acid solution (5%) was added to the n-hexane solution to quench the reaction. The reaction liquid was removed to give an oil-like polymer. The catalytic activity was 9.4×10⁶ g/(mol_{Ni}.h) and the oil-like polyethylene had a weight-average molecular weight of 2156 g/mol, a branching degree of 169 CH₃/1000 C and a bromine number of 32.4.

### Application Example 35 Ethylene Polymerization

A 2 L polymerizer was heated to 100° C., underwent 3 cycles of vacuum and filling with N₂ and cooled to 60° C. Under nitrogen atmosphere, 1.2 L of n-hexane as a solvent, a solution (20 mL, 1 mol/L) of diethylaluminium chloride as a cocatalyst in toluene and the complex C55 (25 μmol) were added polymerizing for 30 min under an ethylene pressure of 5 atm. The ethylene was cut off and 1 mL of methanolic hydrochloric acid solution (5%) was added to the n-hexane solution to quench the reaction. The reaction liquid was removed to give an oil-like polymer. The catalytic activity was 1.3×10⁷ g/(mol_{Ni}.h) and the oil-like polyethylene had a weight-average molecular weight of 3747 g/mol and a branching degree of 143 CH₃/1000 C.

### Application Example 36 Ethylene Polymerization

A 2 L polymerizer was heated to 100° C., underwent 3 cycles of vacuum and filling with N₂ and cooled to 70° C. Under nitrogen atmosphere, 1.2 L of n-hexane as a solvent, a solution (20 mL, 1 mol/L) of diethylaluminium chloride as a cocatalyst in toluene and the complex C55 (25 μmol) were added polymerizing for 30 min under an ethylene pressure of 2 atm. The ethylene was cut off and 1 mL of methanolic hydrochloric acid solution (5%) was added to the n-hexane solution to quench the reaction. The reaction liquid was removed to give an oil-like polymer. The catalytic activity was 9.7×10⁷ g/(mol_{Ni}.h) and the oil-like polyethylene had a weight-average molecular weight of 2676 g/mol and a branching degree of 168 CH₃/1000 C.

### Application Example 37 Oil Product Hydrogenation

To a 250 mL reaction vessel, 100 mL of the polymer solution obtained in the Application Example 34 and 0.3 g of palladium on carbon were added. The reaction vessel was sealed, underwent many cycles of vacuum and filling with inert gas, then underwent 3 cycles of vacuum and filling with H₂, heated to 50° C. and stirred for 6 h. After removing the catalyst and solvent, the hydrogenated product was washed with water, dried and tested obtaining a bromine number of 0.01 and a kinematic viscosity (100° C.) of 45 mm²/s.

### Comparative Example

5 mmol of acenaphthequinone, 13 mmol of 2,6-diisopropylaniline, 42 mL of ethanol and 0.1 g of p-toluenesulfonic acid were added to a 100 mL reaction vial and heated to reflux for 12 h to give a acenaphthequinone diimine ligand with a yield of 82%.

The acenaphthequinone diimine compound obtained above was used to replace the compound L11 of the Example 3-1 and similar procedures to Example 3-1 were conducted to give a reddish-brown acenaphthequinone diimine nickel complex with a yield of 89%.

The acenaphthequinone diimine nickel complex obtained above was used to replace the complex C11 of the Application Example 4 and similar procedures to the example were conducted. The catalytic activity was 1.52×10⁵ g/(mol_{Ni}.h) at 60° C. The polymer obtained had a weight-average molecular weight of 153 Kg/mol, a branching degree of 189 CH₃/1000 C and had no a sharp melting point indicated by a DSC test.

Unless otherwise defined, the terms used in the present disclosure have the common meanings understood by those skilled in the art.

The embodiments described herein are for illustrative purposes only, and are not intended to limit the scope of the disclosure, and those skilled in the art can make various alternatives, changes and modifications within the scope of the disclosure. The disclosure is not limited to the above embodiments and is only limited by the appended claims.

What is claimed is:

1. A compound represented by the following structural formula: 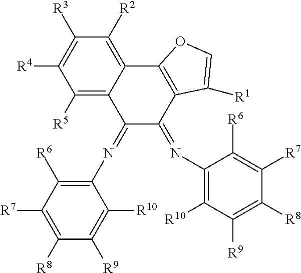
wherein R¹ to R¹⁰ are the same or different and each independently represent a group comprising 1 to 16 carbon atoms selected from hydrocarbyl, substituted hydrocarbyl, alkoxy, alkylthio, alkylamino, haloalkylthio, haloalkoxy, haloalkylamino, aryloxy, arylthio, arylamino; or a group selected from hydrogen, diphenylphosphino, halogen, nitro, and nitrile.

2. The compound according to claim 1, wherein the hydrocarbyl comprises alkyl, cycloalkyl, aryl and aralkyl; and the substituted hydrocarbyl comprises haloalkyl, sulfur-containing alkyl, substituted cycloalkyl, substituted aryl and substituted aralkyl.

3. The compound according to claim 2, wherein the substituents of the substituted aryl, substituted aralkyl and substituted cycloalkyl are selected from C₁-C₄ alkyl, C₁-C₄ haloalkyl, halogen, nitro, or nitrile.

4. The compound according to claim 1, wherein R¹ to R⁵ are each independently selected from the following group of hydrogen, C₁-C₄ alkyl, —OR^{1′}, —SR^{2′}, —NHR^{3′}, —N(R^{4′})₂, substituted or unsubstituted phenyl, benzyl, substituted or unsubstituted cycloalkyl and halogen; and R^{1′}, R^{2′}, R^{3′} R^{4′} are each independently selected from C₁-C₄ alkyl or C₁-C₄ haloalkyl.

5. The compound according to claim 1, wherein R¹ is selected from hydrogen, methyl, ethyl, isopropyl, halogen, phenyl, benzyl, benzhydryl or cycloalkyl; R² is selected from hydrogen, methyl or halogen; R³ is selected from hydrogen, methyl or halogen; R⁴ is selected from hydrogen, methyl, isopropyl, phenyl, benzyl or halogen; and R⁵ is selected from hydrogen, methyl, ethyl, isopropyl or halogen.

6. The compound according to claim 1, wherein R⁶ to R¹⁰ are independently selected from hydrogen, C₁-C₈ alkyl, C₁-C₈ haloalkyl, C₁-C₈ cycloalkyl, halogen, nitro, nitrile, —OR^{1′}, —SR^{2′}, —CH₂SR^{2′}, —NHR^{3′}, —N(R^{4′})₂, phenyl, benzyl, benzhydryl or diphenylphosphino; and R^{1′}, R^{2′}, R^{3′} and R^{4′} are each independently selected from C₁-C₈ alkyl, C₁-C₈ haloalkyl, phenyl or C₁-C₈ substituted phenyl.

7. The compound according to claim 6, wherein R⁶ and R¹⁰ are independently selected from hydrogen, methyl, ethyl, isopropyl, phenyl, benzyl, benzhydryl or cyclohexyl; R⁷ and R⁹ are independently selected from hydrogen, methyl, ethyl or isopropyl; and R⁸ is selected from hydrogen, methyl, ethyl, isopropyl, phenyl, benzyl, benzhydryl or halogen.

8. A preparation method of the compound according to any one of claims 1 to 7 comprising:
carrying out a furan cyclization reaction between a 2-hydroxy-1,4-quinone compound and a haloketone or haloaldehyde to obtain a diketone compound; and
carrying out a ketoamine condensation reaction between the diketone compound and aniline or a substituted aniline to obtain the compound.

9. The preparation method according to claim 8, wherein the catalyst used in the furan cyclization reaction is pyridine, triethylamine or organic ammonium salt; and/or
the temperature of the furan cyclization reaction is 70° C. to 160° C.; and/or
the temperature of the ketoamine condensation reaction is 60° C. to 120° C.; and/or
the catalyst used in the ketoamine condensation reaction is one or more selected from p-toluenesulfonic acid, acetic acid, formic acid and trifluoromethanesulfonic acid; and the catalyst has an additive amount of 0.01% to 30%, based on the mole number of the diketone compound.

10. A complex represented by the following structural formula: 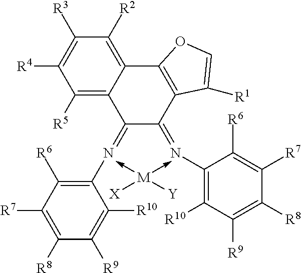
wherein M is selected from Fe, Ni or Pd;
X and Y are independently selected from halogen, C₁-C₄ alkyl or C₂-C₆ alkenyl;
R¹ to R¹⁰ are the same or different and each independently represent a group comprising 1 to 16 carbon atoms selected from hydrocarbyl, substituted hydrocarbyl, alkoxy, alkylthio, alkylamino, haloalkylthio, haloalkoxy, haloalkylamino, aryloxy, arylthio, arylamino; or a group selected from hydrogen, diphenylphosphino, halogen, nitro, and nitrile.

11. A preparation method of the complex according to claim 10 comprising subjecting the compound according to any one of claims 1 to 7 to react with a metal salt to obtain the complex.

12. The preparation method according to claim 11, wherein the metal salt is one or more selected from FeCl₂, NiCl₂, NiBr₂, NiI₂, (DME)NiBr₂, (DME)NiCl₂, PdCl₂, PdBr₂, Pd(OAc)₂, Pd(OTf)₂ and (COD)PdMeCl.

13. A catalyst composition comprising a main catalyst and a cocatalyst, wherein the main catalyst comprises the complex according to claim 10 and the cocatalyst is one or more selected from alkyl aluminoxane, aluminum alkyl and halogenated aluminum alkyl.

14. The catalyst composition according to claim 13, wherein the cocatalyst is one or more selected from methylaluminoxane, ethylaluminoxane, trimethylaluminium, triethylaluminum, triisobutylaluminium, tri-n-butylaluminium, tri-n-hexylaluminium, tri-n-pentylaluminium, tri-n-octylaluminium, diethylaluminium chloride, ethyl aluminum dichloride and ethylaluminum sesquichloride.

15. A use of the catalyst composition according to claims 13 or 14 as catalyst of olefin polymerization.
